(19) United States
(12) Patent Application Publication
(10) Pub. No.: US 2024/0000303 A1
(43) Pub. Date: Jan. 4, 2024

(54) ENDOSCOPE DEVICE, LIGHT SOURCE DEVICE, ENDOSCOPE SYSTEM, AND LIGHT SOURCE CONTROL METHOD

(71) Applicant: OLYMPUS MEDICAL SYSTEMS CORP., Tokyo (JP)

(72) Inventors: Takeshi ITO, Hachioji-shi (JP); Susumu HASHIMOTO, Hachioji-shi (JP); Ryuichi YAMAZAKI, Hachioji-shi (JP); Ryu OTA, Hachioji-shi (JP); Makoto TSUNAKAWA, Hachioji-shi (JP); Jumpei ISHIKAWA, Hachioji-shi (JP); Nobuyoshi ASAOKA, Hachioji-shi (JP); Masahiro NISHIO, Hachioji-shi (JP); Kazuto FUJIWARA, Hachioji-shi (JP); Hideyuki NAGAOKA, Hachioji-shi (JP); Masato TODA, Hachioji-shi (JP)

(73) Assignee: OLYMPUS MEDICAL SYSTEMS CORP., Tokyo (JP)

(21) Appl. No.: 18/367,767

(22) Filed: Sep. 13, 2023

Related U.S. Application Data
(63) Continuation of application No. PCT/JP2021/010329, filed on Mar. 15, 2021.

(51) Int. Cl. A61B 1/06; A61B 1/00; A61B 1/07

(52) U.S. Cl. CPC A61B 1/0655; A61B 1/00006; A61B 1/07; A61B 1/00097

(57) ABSTRACT

An endoscope device includes a processor configured to calculate light source control information based on first information regarding a scope and second information regarding a light source device, and to control a light source of the light source device based on the light source control information. The second information includes a ratio of an amount of light emitted from a distal end of a reference scope to an amount of light emitted from the light source device to the reference scope.

### CROSS REFERENCE TO RELATED APPLICATIONS

This application is a continuation application of PCT Application No. PCT/JP2021/010329 filed on Mar. 15, 2021, the entire contents of which are incorporated herein by reference.

### BACKGROUND

The present application relates to an endoscope device, a light source device, an endoscope system, and a light source control method.

A general medical endoscope system can use various scopes (endoscopes), various light source devices, and various video processors (video signal processing devices) in combination. The light source device and the video processor are configured integrally or separately. In addition, an endoscope system generally has so-called compatibility that can be used with a scope, a light source device, and a video processor over a release period and a generation.

As the scope, various models are used according to an observation site and application. For example, as the scope, scopes having various diameters and functions are used according to the observation site, a use purpose, and the like, and imaging elements and illumination optical systems (light guides and illumination lenses) provided in the scopes have various specifications. For this reason, a color balance of the illumination light emitted from the scope, an upper limit value of a light amount, and the like are different depending on the scope.

Furthermore, light source devices and video processors having various functions are also used. For example, as the light source device, various types of light sources are used depending on models and generations. While lamp light sources such as xenon and halogen have been used for some time, use light sources using semiconductor light sources such as LEDs and lasers have been increasing recently. Characteristics of the illumination light emitted from the light source device, a ratio of the illumination light incident on the scope, and the like are also different depending on the light source device. In addition, an image processing function, a color adjustment function, a noise canceling function, and the like included in the combined video processor are also different depending on the video processor.

Therefore, the upper limit value of the amount of light that can be emitted from a distal end of the scope, the optimum color balance, a gain at the time of image processing, and the like are different depending on the combination of the scope, the light source device, and the video processor. Therefore, in a so-called compatible endoscope system in which various scopes, various light source devices, and various video processors can be combined, it is desired to realize optimum operations, functions, restrictions, and the like in each combination.

JP 2009-213742A proposes an endoscope light source device that reads scope identification information from an endoscope, refers to a light source control information table based on the scope identification information, acquires light source control information corresponding to a connected endoscope, and controls a light amount adjustment mechanism based on the light source control information to adjust the light amount of the light source so that the light amount becomes equal to or less than a limit value.

### SUMMARY

According to an aspect of the present application, there is provided an endoscope device including a processor configured to: calculate light source control information based on first information regarding a scope and second information regarding a light source device; and control a light source of the light source device based on the light source control information, in which the second information includes a ratio of an amount of light emitted from a distal end of a reference scope to an amount of light emitted from the light source device to the reference scope.

According to another aspect of the present application, there is provided a light source device including a processor configured to: calculate light source control information based on first information regarding a scope and second information regarding the light source device; and control a light source of the light source device based on the light source control information, in which the second information includes a ratio of an amount of light emitted from a distal end of a reference scope to an amount of light emitted from the light source device to the reference scope.

According to still another aspect of the present application, there is provided an endoscope system including a scope and a light source device, the endoscope system including a processor configured to: calculate light source control information based on first information regarding the scope and second information regarding the light source device; and control a light source of the light source device based on the light source control information, in which the second information includes a ratio of an amount of light emitted from a distal end of a reference scope to an amount of light emitted from the light source device to the reference scope.

According to still another aspect of the present application, there is provided a light source control method including: calculating light source control information based on first information regarding a scope and second information regarding a light source device; and controlling a light source of the light source device based on the light source control information, in which the second information includes a ratio of an amount of light emitted from a distal end of a reference scope to an amount of light emitted from the light source device to the reference scope.

### BRIEF DESCRIPTION OF THE DRAWINGS

FIG. 1 is a diagram illustrating a configuration of an endoscope system according to an embodiment.

FIG. 2 is a diagram illustrating first information, second information, and combination information according to the embodiment of FIG. 1.

FIG. 3 is a diagram illustrating first information, second information, and combination information according to an embodiment.

FIG. 4 is a diagram illustrating first information, second information, and combination information according to an embodiment.

FIG. 5 is a diagram illustrating first information, second information, and combination information according to an embodiment.

FIG. 6 is a diagram illustrating a configuration of an endoscope system according to an embodiment.

FIG. 7 is a diagram illustrating third information according to the embodiment of FIG. 6.

FIG. 8 is a diagram illustrating a configuration of an endoscope system according to an embodiment.

FIG. 9 is a diagram illustrating a configuration of an endoscope system according to an embodiment.

FIG. 10 is a diagram illustrating first information, second information, and combination information according to an embodiment.

### DETAILED DESCRIPTION

In a case where the light source control information for each endoscope is stored in a memory of the light source device as in JP 2009-213742 A, it is extremely difficult to assume specifications of an endoscope to be developed in the future. In addition, in an existing endoscope, a specification change such as a change of an illumination optical system or a correction of a heat dissipation function may be performed, and it is extremely difficult to assume such a specification change.

Hereinafter, embodiments of the present application will be described with reference to the drawings.

### First Embodiment

An endoscope system according to a first embodiment includes a scope, and a light source device and a video processor integrally or separately configured. Note that one or both of the light source device and the video processor are also referred to as an endoscope device.

In the endoscope system according to the first embodiment, information necessary for realizing an optimum operation, function, restriction, and the like in a combination of the scope, the light source device, and the video processor is classified into scope specific information, light source device specific information, video processor specific information, and combination information.

The scope specific information includes information regarding an individual of the scope, such as a serial number, a use start date, an inspection status, an accumulated use count, and a repair history of the scope, and information such as adjustment information at the time of manufacturing, a manufacturing factory, and a shipping date. The information can be used for checking an inspection time and analyzing when a failure or the like occurs. In addition, the scope specific information also includes information such as a type (a difference in spectral sensitivity or the like) of an imaging element included in the scope, a spectral transmittance and a diameter (the number of optical fiber wires) of the light guide, light distribution of the emitted light, and an upper limit value of an amount of light that can be emitted.

The light source device specific information includes information regarding an individual of the light source device such as a serial number of the light source device, a use start date, an inspection status, an accumulated use time, and a repair history, and information such as adjustment information at the time of manufacturing, a manufacturing factory, and a shipping date. The information can be used for checking an inspection time and analyzing when a failure or the like occurs. In addition, the light source device specific information also includes information such as a type (a difference between a lamp, an LED, and a laser, a difference in spectral light amount accompanying the difference, or the like) of a light source included in the light source device, coupling efficiency (a ratio at which the amount of light emitted from the light source device is incident on the light guide) with the light guide.

The video processor specific information includes information regarding an individual of the video processor, such as a serial number, a use start date, an inspection status, an accumulated use time, and a repair history of the video processor, and information such as adjustment information at the time of manufacturing, a manufacturing factory, and a shipping date. The information can be used for checking the inspection time and analyzing when a failure or the like occurs. Furthermore, the video processor specific information also includes information such as an upper limit value of a gain at the time of image processing of the video processor.

Note that the light source device and the video processor are often used in a specific combination, and integrated ones are also widely used. Therefore, the light source device specific information and the video processor specific information can be shared or handled as the same information.

The combination information is control information for determining numerical values and conditions related to operation, functions, restrictions, and the like of the endoscope system in a combination of the scope, the light source device, and the video processor. The combination information is derived using two or more of information (also referred to as combination scope information or first information) depending on the model, generation, or the like of the scope, information (also referred to as combination light source device information or second information) depending on the model, generation, or the like of the light source device, and information (also referred to as combination video processor information or third information) depending on the model, generation, or the like of the video processor. Note that the combination scope information is information dependent on the scope and is included in the scope specific information. The combination light source device information is information depending on the light source device and is included in the light source device specific information. The combination video processor information is information dependent on the video processor and is included in the video processor specific information. Specifically, the combination scope information includes information such as a type (a difference in spectral sensitivity or the like) of the imaging element included in the scope, a spectral transmittance and a diameter (the number of optical fiber wires) of the light guide, light distribution of the emitted light, and the upper limit value of the amount of light that can be emitted. The combination light source device information includes information such as a type (a difference between a lamp, an LED, and a laser, a difference in a spectral light amount accompanying the difference, and the like) of a light source included in the light source device, coupling efficiency (ratio at which a light amount emitted from the light source is incident on the light guide) with the light guide. The combination video processor information includes information such as an upper limit value of a gain at the time of image processing of the video processor.

The combination information derived using two or more of the combination scope information, the combination light source device information, and the combination video processor information is, for example, information regarding an emission light amount upper limit value from the light source device that satisfies the upper limit value of the amount of light that can be emitted from the distal end of the scope by the scope, an optimum color balance of the illumination light, an upper limit value of a gain of auto gain control (AGC) included in the video processor, and the like.

Note that the scope specific information, the light source device specific information, and the video processor specific information are desirably stored so as to be usable when necessary. For example, the scope specific information can be stored in a memory included in the scope. The light source device specific information can be stored in a memory included in the light source device. The video processor specific information can be stored in a memory included in the video processor. Alternatively, the scope specific information, the light source device specific information, and the video processor specific information may be stored in a memory accessible from the scope, the light source device, and the video processor. In this case, the storage location may be placed on a network, provided by a cloud (cloud computing), or the like. The combination scope information, the combination light source device information, and the combination video processor information may also be stored in a storage destination similar to the scope specific information, the light source device specific information, and the video processor specific information, or may be stored in another storage destination.

In the endoscope system according to the first embodiment, an information calculation unit included in the light source device or the video processor derives the combination information by using two or more of the combination scope information, the combination light source device information, and the combination video processor information, and control (for example, light source control) is performed based on the combination information. Note that the information used to derive the combination information needs to be stored in one or a plurality of memories accessible directly or indirectly from the information calculation unit.

The combination information is derived when the combination of the scope, the light source device, and the video processor is determined. For example, when the scope, the light source device, and the video processor are electrically or mechanically connected, the combination information is derived. Note that details of derivation of the combination information will be described later.

Alternatively, combination information for each combination of the scope, the light source device, and the video processor may be derived and stored in advance, and the corresponding combination information may be read when the combination of the scope, the light source device, and the video processor is determined. For a combination that has been connected once, combination information derived at the time of connection may be stored, and when the combination of the scope, the light source device, and the video processor is determined, the corresponding combination information may be read. In this case, the combination information for each combination is preferably stored in a memory included in the light source device or the video processor. Alternatively, the information may be stored in a memory directly or indirectly accessible from an information calculation unit included in the light source device or the video processor, or may be stored in a memory on a network or a memory provided by a cloud.

According to the first embodiment, it is possible to realize optimum operation, functions, restrictions, and the like in various combinations of the scope, the light source device, and the video processor in the endoscope system.

Specific examples of the first embodiment will be described as the following embodiments.

### Second Embodiment

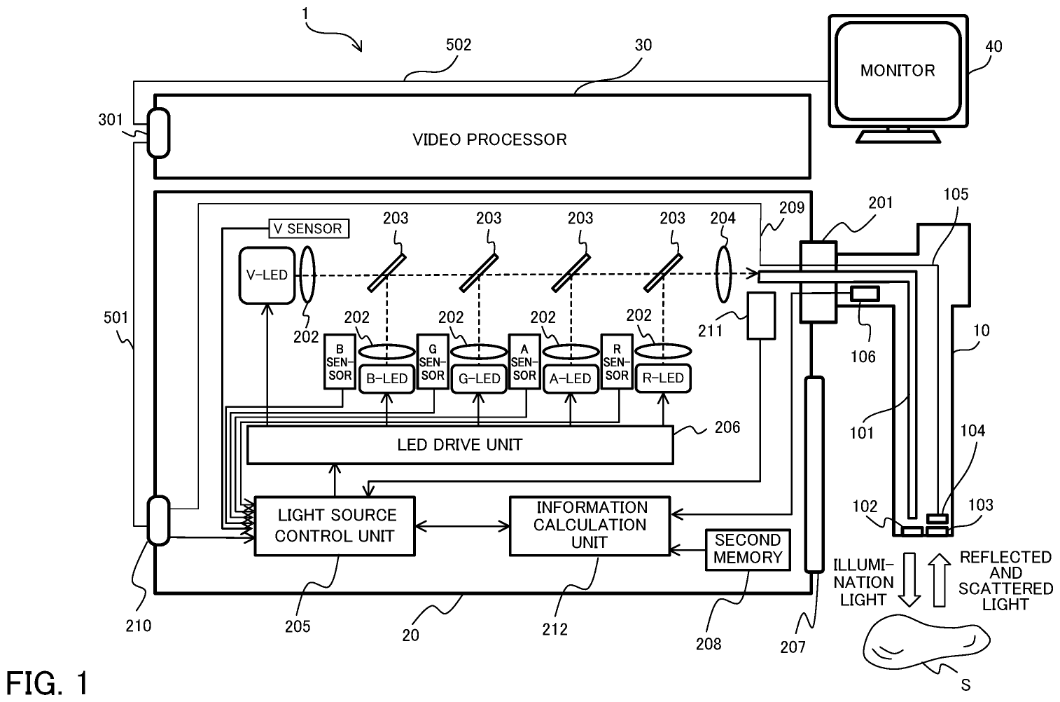

FIG. 1 is a diagram illustrating a configuration of an endoscope system according to a second embodiment.

An endoscope system 1 illustrated in FIG. 1 includes a scope 10, a light source device 20, a video processor 30, and a monitor 40. The scope 10 is configured to be detachable from the light source device 20 with a scope connector 201. Note that the endoscope system 1 can connect various scopes (including the scope 10) and the light source device 20 according to an observation purpose or the like, can connect the scope 10 and various light source devices (including the light source device 20), and has so-called compatibility.

The scope 10 includes a light guide 101 and an illumination lens 102, guides LED light emitted from the light source device 20 to the distal end of the scope by the light guide 101, and irradiates a subject S with the LED light as illumination light via the illumination lens 102. Furthermore, the scope 10 includes an imaging lens 103 and an imaging element 104 at the distal end of the scope for imaging the subject S, and captures an optical image (reflected and scattered light) of the subject S irradiated with the illumination light. The imaging element 104 converts the captured image into an electric signal, and transmits the electric signal to the video processor 30 via the light source device 20 by means of a video signal line 105 as a video signal.

Furthermore, the scope 10 includes a first memory 106 that stores scope specific information that is information regarding the scope 10. The scope specific information includes first information (combination scope information) that is information depending on the model, generation, or the like of the scope 10.

In addition, the scope 10 includes components included in a general scope, such as a wire for curving the distal end portion of the scope, an air/water supply pipe for sending air or water to an observation target site, and an operation unit provided with a handle, switches, and the like for curving the distal end portion of the scope. However, these components are not illustrated in FIG. 1 for convenience.

The light source device 20 includes a V-LED (purple), a B-LED (blue), a G-LED (green), an A-LED (amber), and an R-LED (red) as LEDs (LED light sources) of five colors having different wavelengths. The LED light emitted from each LED is substantially collimated by a collimating lens 202, multiplexed by a dichroic mirror 203, and emitted to an incident end of the light guide 101 of the scope 10 via the condensing lens 204. Light emission of each LED is controlled by the light source control unit 205. An optical sensor (V sensor, B sensor, G sensor, A sensor, R sensor) is installed in the vicinity of each LED, and detects a part of light emitted from the LED. Each optical sensor outputs an electric signal (light amount signal) corresponding to the amount of the received light to the light source control unit 205. The light source control unit 205 is electrically connected to an LED drive unit 206, and controls the LED drive unit 206 so as to supply a current corresponding to the light amount signal received from each optical sensor to each LED.

Furthermore, the light source device 20 includes a second memory 208 that stores light source device specific information that is information regarding the light source device 20. The light source device specific information includes the second information (combination light source device information) that is information depending on the model, generation, or the like of the light source device 20.

The light source device 20 further includes an operation panel 207 used for an input operation of a user, a connection detection unit 211 that detects that the scope 10 is connected to the light source device 20, an information calculation unit 212 described later, and the like.

The video processor 30 includes an image processing circuit (not illustrated) and processes an image signal (video signal) acquired by the scope 10. Note that the image signal from the imaging element 104 of the scope 10 is transmitted to an image processing circuit (not illustrated) via the video signal line 105 of the scope 10, the scope connector 201 of the light source device 20, the video signal line 209, an electrical connector 210, an electrical wire 501, and an electrical connector 301 of the video processor 30.

The image signal processed by the video processor 30 is transmitted to the monitor 40 via the electrical connector 301 and a monitor signal line 502. The monitor 40 displays an image based on the received image signal.

Furthermore, the image signal processed by the video processor 30 is subjected to appropriate processing and transmitted as a luminance signal indicating brightness of the image to the light source control unit 205 of the light source device 20 via the electrical connector 301, the electrical wire 501, and the electrical connector 210. The light source control unit 205 receives a luminance signal from the video processor 30, sets a target light amount that is a light source light amount with which an image has appropriate brightness, calculates a driving current based on the target light amount and an output from each optical sensor, and outputs the driving current to the LED drive unit 206. That is, the light amount of the LED light emitted from each LED is detected by an optical sensor installed in the vicinity of each LED, and is feedback-controlled by the light source control unit 205.

In the endoscope system 1, when the scope 10 is connected to the light source device 20, the connection detection unit 211 provided in the vicinity of the scope connector 201 detects the connection, and transmits an intent of the connection to the light source control unit 205. Upon receiving this, the light source control unit 205 requests the information calculation unit 212 for combination information in the combination of the connected scope 10 with the light source device 20 and the video processor 30. The information calculation unit 212 that has received the request for the combination information reads the first information from the first memory 106 of the scope 10 and reads the second information from the second memory 208 of the light source device 20. Then, the combination information is derived from the first information and the second information. In the second embodiment, the emission light amount upper limit value (an example of light source control information) of the light source device 20 satisfying the upper limit value of the amount of light that can be emitted from the distal end of the scope by the scope 10 is derived as the combination information and transmitted to the light source control unit 205. Upon receiving this, the light source control unit 205 sets the upper limit value of the amount of light of each LED according to the combination information (the emission light amount upper limit value of the light source device 20), and controls each LED based on the upper limit value.

Here, derivation of the combination information performed by the information calculation unit 212 will be specifically described.

The information calculation unit 212 reads, from the first memory 106 of the scope 10, the light amount upper limit value (P.S.limit.s) that is the first information and that can be emitted from the distal end of the scope, and a ratio (ηs/ηss) of transmittance (ns) of the LED light in the light guide optical system included in the scope 10 to transmittance (ηss) of the LED light in the light guide optical system included in the reference scope. In addition, the information calculation unit 212 reads, from the second memory 208 of the light source device 20, a ratio (P.S.ss/P.B.ss) of the amount of light (P.S.ss) emitted from the distal end of the scope of the reference scope to the amount of light (P.B.ss) emitted from the light source device 20 to the reference scope, which is the second information. Then, using the read first information and second information, the information calculation unit 212 derives an emission light amount upper limit value (P.B.limit.s) of the light source device 20 satisfying the upper limit value of the amount of light (P.S.limit.s) that can be emitted from the distal end of the scope by the scope 10 according to the following Expression (1).

P.B.limit.s=P.S.limit.s/(ηs/ηss)/(P.S.ss/P.B.ss)=P.S.limit.s×(ηss/ηs)×(P.B.ss/P.S.ss) Expression (1)

Note that the reference scope described above is preferably, for example, a scope that is frequently used and is used as a standard. Alternatively, a scope of a higher-order model (flag-ship model) capable of using all observation modes, a scope of a large-light quantity model equipped with a large-diameter light guide capable of emitting the brightest illumination light, and the like are also suitable. In addition, the above-described P.B.ss and P.S.ss are not limited to the maximum light amount, but since the ratio (P.S.ss/P.B.ss) may change depending on the light amount, it is desirable that the light amount be close to the maximum light amount. However, this is not the case as long as the change in ratio depending on the light amount is not large.

Further, assuming a case where the above-described P.S.limit.s is different for each observation mode such as a white light imaging (WLI) mode, a narrow band imaging (NBI) mode, and an enlarged observation mode, the first information may include the P.S.limit.s for each observation mode. For example, P.S.limit.s.w as P.S.limit.s in the case of the WLI mode, P.S.limit.s.n as P.S.limit.s in the case of the NBI mode, P.S.limit.s.w.mag as P.S.limit.s in the case of the enlarged observation WLI mode, and P.S.limit.s.n.mag as P.S.limit.s in the case of the enlarged observation NBI mode may be included. In this case, for example, the information calculation unit 212 may derive P.B.limit.s using P.S.limit.s according to the observation mode after the switching at the switching timing of the observation mode (or until the switching timing of the observation mode), and transmit the P.B.limit.s to the light source control unit 205. Then, the light source control unit 205 may set the upper limit value of the amount of light of each LED according to the P.B.limit.s and control each LED based on the upper limit value. In this case, the above-described reference scope may be common or different in all observation modes. However, in a case where different reference scopes are used for each observation mode, it is necessary to use a value corresponding to the reference scope according to the observation mode as P.S.ss/P.B.ss used for deriving P.B.limit.s. For such a case, the second information may include P.S.ss/P.B.ss for each observation mode.

In addition, instead of the reference scope described above, an optical system that simulates the reference scope such as a single light guide or a rod lens may be used. In this case, a value depending on the simulated optical system is used for the above-described P.S.ss/P.B.ss.

As described above, according to the second embodiment, by storing P.S.limit.s and ηs/ηss as the first information in the first memory 106 and storing P.S.ss/P.B.ss as the second information in the second memory 208, it is possible to appropriately control the amount of light that can be emitted from the distal end of the scope 10 in the combination of the scope 10 and the light source device 20.

In the second embodiment, since the relationship of P.S.ss=ηss×P.B.ss is established, the above Expression (1) can be modified as the following Expression (2).

P.B.limit.s=P.S.limit.s/ηs Expression (2)

With such a deformation, it is possible to derive the P.B.limit.s only with the ns and the P.S.limit.s (in other words, only with the first information), but it is difficult to determine the ns without specifying the light source device 20, and there may be a problem that a difference depending on a difference in the light source device (for example, a difference in an emission light beam diameter of the light source device) is large. In the second embodiment, the accuracy of P.B.limit.s can be improved by using ηss as a reference.

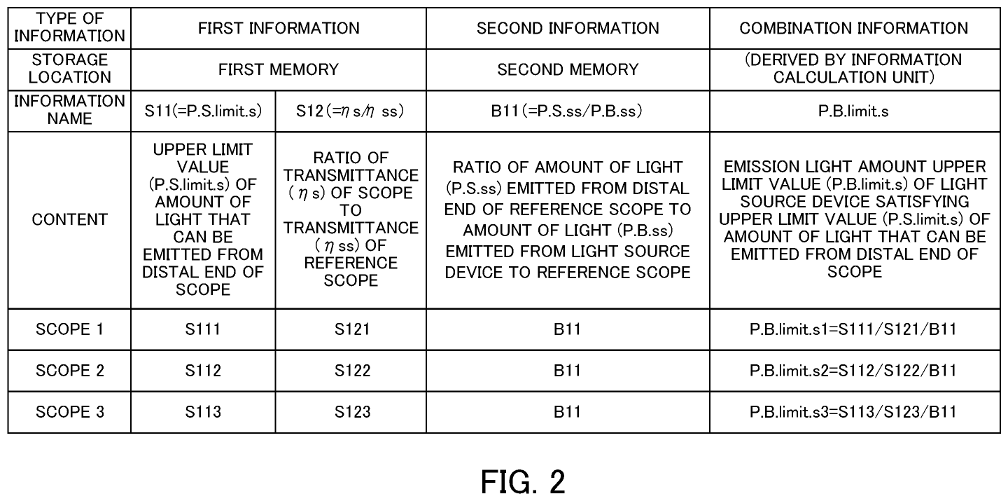

FIG. 2 is a diagram illustrating the first information, second information, and combination information according to the second embodiment. Here, the first information, the second information, and the combination information in the combination of each of the three types of scopes (“scope 1”, “scope 2”, and “scope 3”) including the scope 10 and the light source device 20 are illustrated. Further, P.S.limit.s and ηs/ηss of the first information are indicated as S11 and S12, S11 and S12 for the “scope 1” are indicated as S111 and S121, S11 and S12 for the “scope 2” are indicated as S112 and S122, and S11 and S12 for the “scope 3” are indicated as S113 and S123. Further, P.S.ss/P.B.ss of the second information is indicated as B11. In this example, for example, the combination information P.B.limit.s (P.B.limit.s1) in the combination of the “scope 1” and the light source device 20 is derived according to the following Expression (3) using S111 and S121 for the “scope 1” and B11 for the light source device 20. Note that the following Expression (3) corresponds to the above Expression (1).

P.B.limit.s1=S111/S121/B11 Expression (3)

Combination information P.B.limit.s (P.B.limit.s2) in the combination of the “scope 2” and the light source device 20 and combination information P.B.limit.s (P.B.limit.s3) in the combination of the “scope 3” and the light source device 20 are similarly derived.

### Third Embodiment

A third embodiment is an aspect in which color balance information (an example of light source control information) is derived as the combination information, and the amount of light of each LED is set according to the color balance information. Hereinafter, the third embodiment will be described, but the description thereof will be made only for differences from the second embodiment, and description of common parts will be omitted.

In the third embodiment, the first information stored in the first memory 106 included in the scope 10 includes a type of a color filter of the imaging element 104 and the information (S21R, S21G, S21B) of light receiving sensitivity of each of the R, G, and B color regions of the imaging element 104. For example, in the case of the imaging element of a primary color Bayer array, information of the light receiving sensitivity, which is the product of the transmittance of each color filter and the light receiving sensitivity of the imaging element, is included as S21R, S21G, and S21B. Alternatively, in the case of a complementary color imaging element, information on transmittance of a color filter (CMY), light receiving sensitivity of the imaging element, and image processing of the imaging element is included as S21R, S21G, and S21B. In this case, information of light receiving sensitivity obtained as a result including image processing combined with a color filter (CMY) may be included as S21R, S21G, and S21B. Alternatively, in the case of the combination of a monochrome imaging element and a sequential light emitting light source, the light receiving sensitivity information in each color region of R, G, and B of the monochrome imaging element is included as S21R, S21G, and S21B.

Here, the R region is a red region and is defined as a light receiving sensitivity region of 585 nm to 780 nm. The G region is a green region, and is defined as a light receiving sensitivity region of 495 nm to 584 nm. The B region is a blue region and is defined as a light receiving sensitivity region of 400 nm to 494 nm.

In addition, the second information stored in the second memory 208 included in the light source device 20 includes information of a standard color balance set from a spectral shape or the like of the LEDs of five colors included in the light source device 20. That is, the amount of light emitted from the LEDs of five colors for each of the R, G, and B color regions described above is included as a ratio of each other. As the color balance, information of R:G:B is included as information of color balance in the case of white and standard color balance of various types of special light. Here, the light of the V-LED and the B-LED is substantially included in the B region, the light of the G-LED is substantially included in the G region, and the light of the A-LED and the R-LED is substantially included in the R region. Note that the color balance is desirably defined not by the light emitted from each LED but by the color balance after passing through the multiplexing optical system such as the dichroic mirror 203, and is more desirably the color balance immediately before entering the light guide of the scope.

The ratio of the color balance is stored as information of the standard color balance in the second memory 208 as a ratio of each of the light amount (B22B) of the B region and the light amount (B22R) of the R region when the light amount of the G region is 1 (since the light amount of the G region is 1, it is not necessary to store the ratio). Note that the optimum color balance ratio varies depending on the light receiving sensitivity of the imaging element. Therefore, the second information stored in the second memory also includes information of the light receiving sensitivity (B21R, B21G, B21B) of the imaging element assumed to be standard, which is used for calculating the standard color balance.

Using the first information and second information, the information calculation unit 212 calculates a color balance (Light amount ratio of R, G, and B) optimal for the connected scope of the illumination light emitted from the light source device 20, which is the combination information. That is, the light intensity ratio (Q.B.Pr.s) of R and the light intensity ratio (Q.B.Pb.s) of B, which are combination information, are calculated and derived according to the following Expressions (4) and (5).

Q.B.Pr.s=B22R×(B21R/S21R)/(B21G/S21G) Expression (4)

Q.B.Pb.s=B22B×(B21B/S21B)/(B21G/S21G) Expression (5)

The above Expressions (4) and (5) are calculated such that the light amount ratio of G (Q.B.Pg.s.)=1.

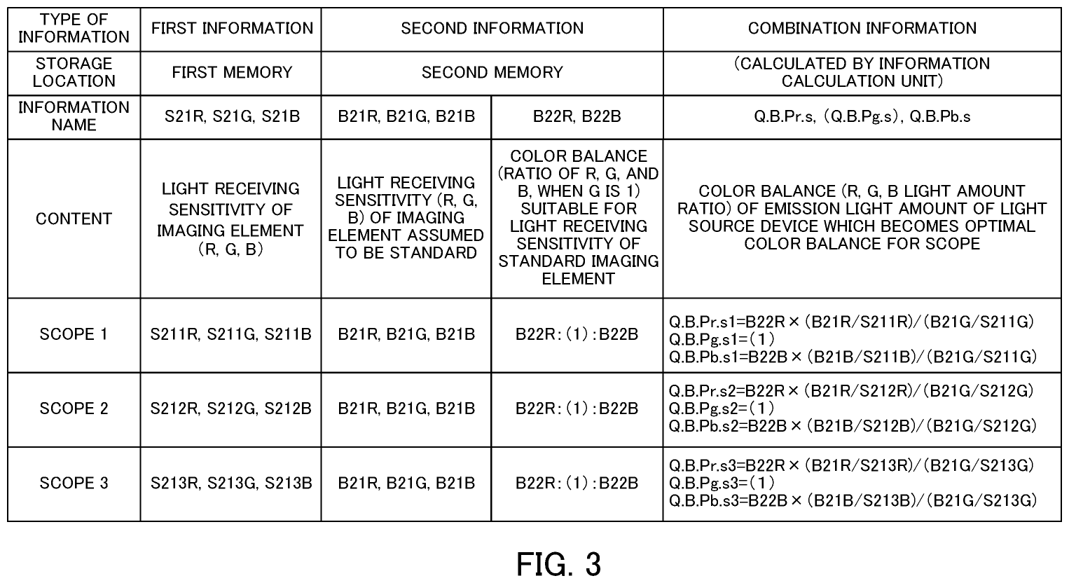

FIG. 3 is a diagram illustrating the first information, second information, and combination information according to the third embodiment. Here, the first information, the second information, and the combination information in the combination of each of the three types of scopes (“scope 1”, “scope 2”, and “scope 3”) including the scope 10 and the light source device 20 are also illustrated. In addition, S21R, S21G, and S21B for the “scope 1” are illustrated as S211 R, S211 G, and S211 B, S21R, S21G, and S21B for the “scope 2” are illustrated as S212 R, S212 G, and S212 B, and S21R, S21G, and S21B for the “scope 3” are illustrated as S213 R, S213 G, and S213 B.

According to the third embodiment, it is possible to realize a color balance optimum for a combination of the scope and the light source device.

In the third embodiment, the first information may include information of S21R, S21G, and S21B for each observation mode, and the second information may include information of B21R, B21G, and B21B and information of B22R and B22B for each observation mode. In this case, for example, at the switching timing of the observation mode (or until the switching timing of the observation mode), the information calculation unit 212 may calculate Q.B.Pr.s and Q.B.Pb.s based on the information of S21R, S21G, and S21B according to the observation mode after the switching included in the first information, and the information of B21R, B21G, and B21B and the information of B22R and B22B according to the observation mode after switching included in the second information.

### Fourth Embodiment

A fourth embodiment is a modification of the second embodiment, in which two pieces of information of P.S.limit.s (S11) and ηs/ηss (S12) included in the first information stored in the first memory of the scope are collectively stored as one piece of information. Specifically, there is an aspect in which two pieces of information of P.S.limit.s (S11) and ηs/ηss (S12) are collectively stored as one piece of information of P.S.limit.s (S11)/(ηs/ηss (S12)), and the others are the same as those in the second embodiment.

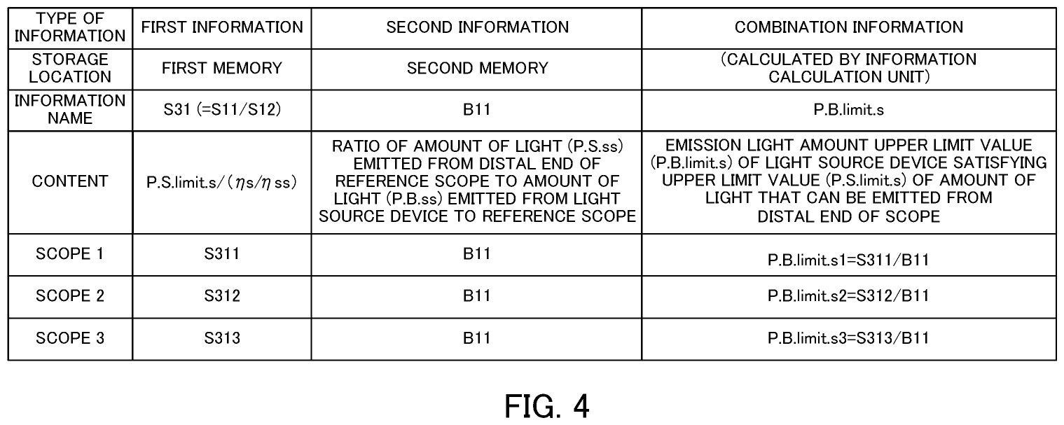

FIG. 4 is a diagram illustrating first information, second information, and combination information according to a fourth embodiment. Note that those illustrated in FIG. 4 correspond to those illustrated in FIG. 2. Here, P.S.limit.s/(ηs/ηss) of the first information is indicated as S31, S31 for the “scope 1” is indicated as S311, S31 for the “scope 2” is indicated as S312, and S31 for the “scope 3” is indicated as S313. In this example, for example, the combination information P.B.limit.s (P.B.limit.s1) in the combination of the “scope 1” and the light source device 20 is derived according to the following Expression (6) using S311 for the “scope 1” and B11 for the light source device 20.

P.B.limit.s1=S311/B11 Expression (6)

Combination information P.B.limit.s (P.B.limit.s2) in the combination of the “scope 2” and the light source device 20 and combination information P.B.limit.s (P.B.limit.s3) in the combination of the “scope 3” and the light source device 20 are similarly derived.

According to the fourth embodiment, it is possible to reduce a storage capacity of the first memory in which the first information is stored, improve the reliability of information management, and simplify the calculation Expression used by the information calculation unit 212.

### Fifth Embodiment

The fifth embodiment is a modification of the fourth embodiment, in which the first information further includes a correction factor α associated with special circumstances of the scope regarding the upper limit value of the amount of light that can be emitted from the distal end of the scope (P.S.limit.s (S11)), and accordingly, a calculation formula used by the information calculation unit 212 to derive the P.B.limit.s is different. The rest is the same as that of the fourth embodiment.

The upper limit value of the amount of light that can be emitted from the distal end of the scope is generally determined depending on the limit (standards such as Japanese Industrial Standards (JIS) and an upper limit of a temperature at which a living body is not damaged) of the temperature of the distal end of the scope, the heat resistance level of the member to be used, and the like. Since members and basic structures used in the scope are substantially common, this upper limit value can be approximately estimated, for example, according to a ratio of the number of optical fiber wires of a light guide included in the scope. However, there is a scope having special circumstances such as a member or a structure is changed or heat dissipation is further improved depending on special circumstances such as an observation target or a mounted function which is an object of the scope. In the case of such a scope, the upper limit value of the amount of light that can be emitted from the distal end of the scope may be greatly different from the other scopes. In order to cope with such a case, in the fifth embodiment, the first information further includes the correction factor α described above. The correction factor α is indicated by a ratio of how high or low the upper limit value of the amount of light that can be emitted from the distal end of the scope is. For example, α=1.1 is obtained when the upper limit value is 10% higher, and α=0.8 is obtained when the upper limit value is 20% lower.

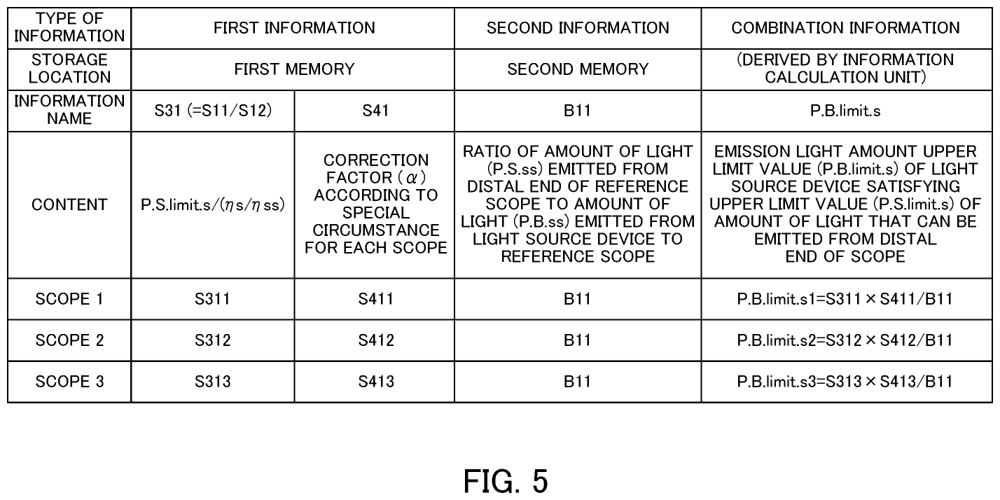

FIG. 5 is a diagram illustrating first information, second information, and combination information according to the fifth embodiment. Note that those illustrated in FIG. 5 correspond to those illustrated in FIG. 4. Here, the correction factor α of the first information is indicated as S41, S41 for the “scope 1” is indicated as S411, S41 for the “scope 2” is indicated as S412, and S41 for the “scope 3” is indicated as S413. In this example, for example, the combination information P.B.limit.s (P.B.limit.s1) in the combination of the “scope 1” and the light source device 20 is derived according to the following Expression (7) using S311 and S411 for the “scope 1” and B11 for the light source device 20.

P.B.limit.s1=S311×S411/B11 Expression (7)

Combination information P.B.limit.s (P.B.limit.s2) in the combination of the “scope 2” and the light source device 20 and combination information P.B.limit.s (P.B.limit.s3) in the combination of the “scope 3” and the light source device 20 are similarly derived.

According to the fifth embodiment, since the first information further includes the correction factor α, the upper limit value of the amount of light that can be emitted from the distal end of the scope can be set to an appropriate value even for scopes having various special circumstances.

In the fifth embodiment, instead of including two pieces of information of S31 (P.S.limit.s/(ηs/ηss)) and S41 (correction factor α), the first information may include one piece of information obtained by multiplying S31 by S41 in advance.

In the fifth embodiment, as a modification of the second embodiment, the first information according to the second embodiment may further include the correction factor α.

### Sixth Embodiment

A sixth embodiment is a modification of the second to fifth embodiments, and is an aspect in which the video processor 30 further includes a third memory that stores third information.

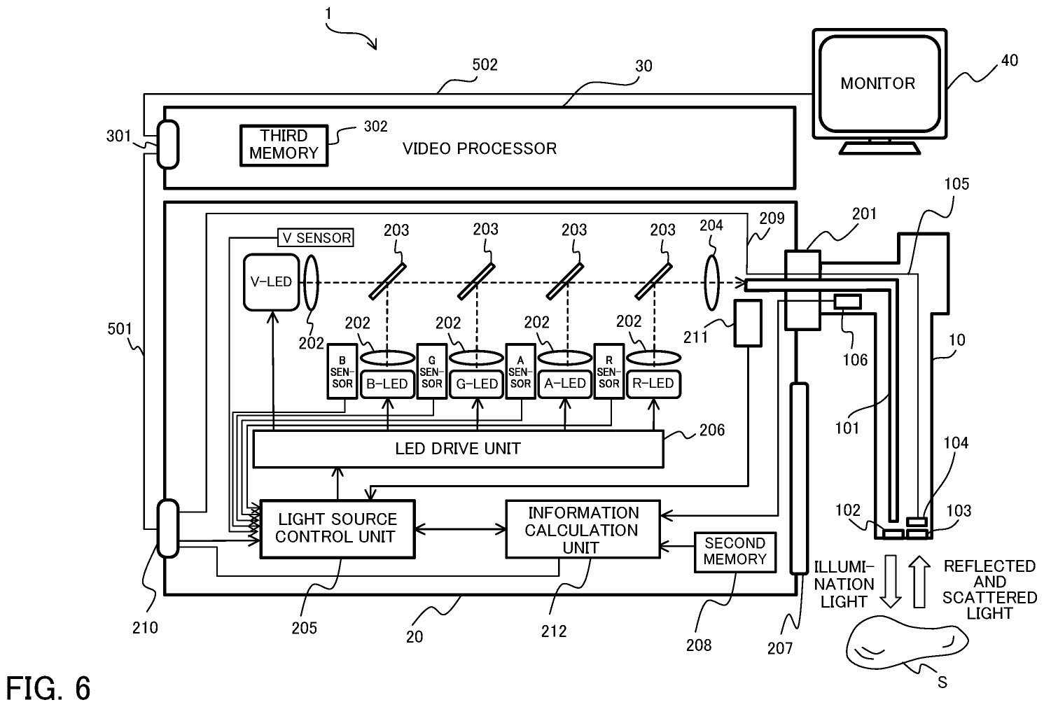

FIG. 6 is a diagram illustrating a configuration of an endoscope system according to a sixth embodiment.

In the endoscope system 1 illustrated in FIG. 6, the video processor 30 includes a third memory 302 in which video processor specific information that is information regarding the video processor 30 is stored. The video processor specific information includes third information (combination video processor information) that is information depending on the model, generation, and the like of the video processor 30. In the sixth embodiment, information on the upper limit value of the gain (the amplification level of the image signal) of the image processing performed by the video processor 30 is included as the third information.

In the image processing performed by the video processor 30, the image signal is amplified for the following reasons. For example, as a result of the light amount emitted by the light source device 20 reaching the upper limit value, the illumination light emitted from the scope 10 is limited, and the reflected/scattered light from the subject S may not reach a level at which an image with sufficient brightness can be obtained. In other words, depending on reflectance of the subject S, the distance between the subject S and the distal end of the scope, and the like, the amount of light incident on the imaging element 104 becomes insufficient, and the luminance of the image signal may decrease. In such a case, the video processor 30 amplifies the image signal and electrically corrects the brightness of the image to be displayed on the monitor 40. The amplification ratio at this time is referred to as a gain, and is expressed by a magnification [times] or a decibel [dB]. Note that the gain in the image processing is a generally handled parameter, and thus a detailed description thereof will be omitted here.

In the endoscope system 1 illustrated in FIG. 6, the video processor 30 is provided with so-called auto gain control (AGC) for amplifying a video signal acquired by the scope 10 with an appropriate gain and displaying an image with appropriate brightness on the monitor 40. Note that, when the gain is excessively increased, a problem such as conspicuous noise on the image displayed on the monitor 40 may occur, and thus, it is general to set an upper limit to the gain. In the sixth embodiment, the third information includes a gain (times or dB) that is an upper limit of the AGC.

The upper limit value of the gain of AGC varies depending on the sensitivity of the imaging element 104, the spectrum pattern of the color filter, the spectrum or light emission pattern of the illumination light, image processing, and the like. Therefore, the gain upper limit value of AGC is combination information determined according to the combination of the scope 10, the light source device 20, and the video processor 30. The third information includes the upper limit value of the gain determined in consideration of the image processing performed by the video processor 30 and the imaging element included in the scope as information for each scope (or each imaging element). The video processor 30 reads and sets the upper limit value of the gain of the corresponding scope included in the third information stored in the third memory 302. Note that which scope is connected to the light source device 20 can be identified by, for example, acquiring a scope identifier included in the scope specific information stored in the first memory of the scope via the information calculation unit 212, the electrical connector 210, the electrical wire 501, and the electrical connector 301.

Accordingly, even in a case where the brightness of the imaging signal is insufficient due to AGC, it is possible to cause the image to be displayed on the monitor 40 as an image with appropriate brightness, and the image is not amplified with a gain high enough to cause problems such as noise. Note that, in a case where sufficient brightness cannot be obtained even by amplification with the maximum gain, the image displayed on the monitor 40 becomes dark. Therefore, in such a case, a bright image can be obtained by the operator performing an operation such as appropriately adjusting the distance between the distal end of the scope and the subject.

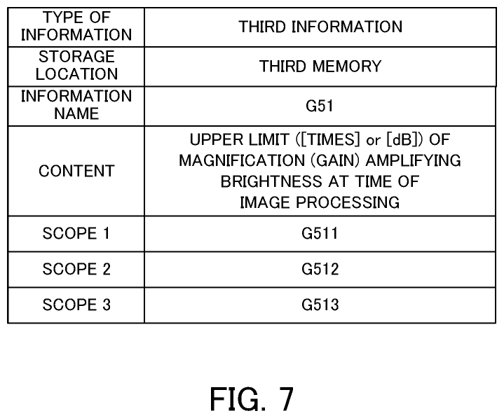

FIG. 7 is a diagram illustrating third information according to the sixth embodiment. Here, the upper limit value (times or dB) of the magnification (gain of AGC) for amplifying the brightness at the time of image processing is indicated as G51, G51 for the “scope 1” is indicated as G511, G51 for the “scope 2” is indicated as G512, and G51 for the “scope 3” is indicated as G513.

According to the sixth embodiment, it is possible to display an image with appropriate brightness and an appropriate noise level on the monitor 40.

### Seventh Embodiment

A seventh embodiment is a modification of each of the second to fifth embodiments, and is an aspect in which the video processor 30 includes the second memory included in the light source device 20.

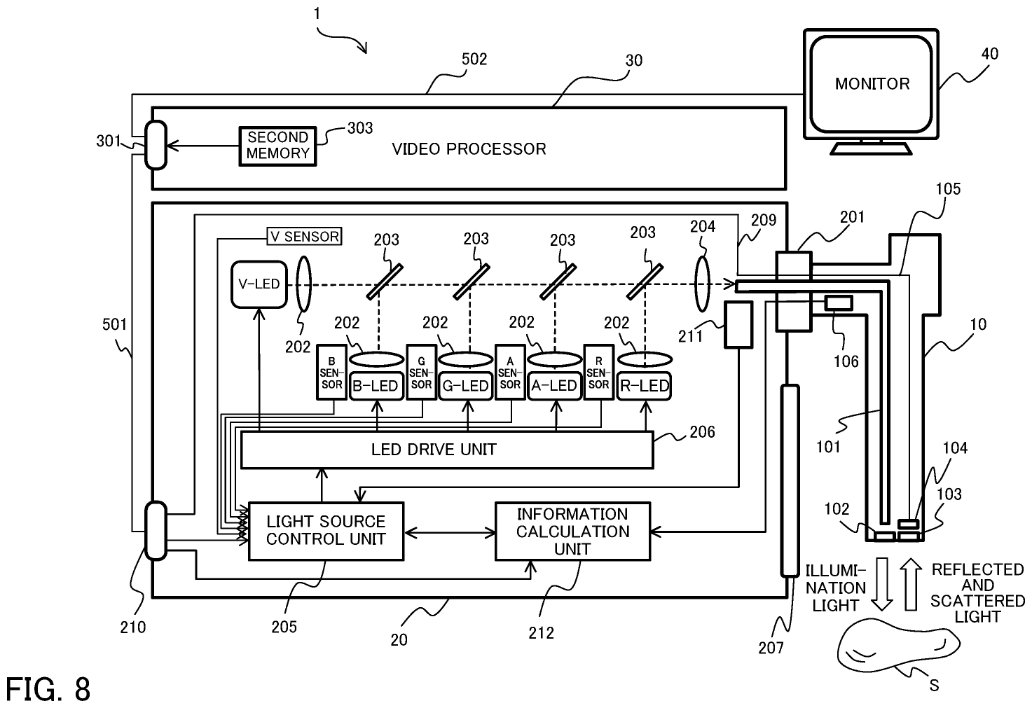

FIG. 8 is a diagram illustrating a configuration of an endoscope system according to a seventh embodiment.

In the endoscope system 1 illustrated in FIG. 8, the video processor 30 includes the second memory 303 instead of the light source device 20 including the second memory. Accordingly, the information calculation unit 212 acquires the second information stored in the second memory 303 included in the video processor 30 via the electrical connector 301, the electrical wire 501, and the electrical connector 210.

Note that, in the seventh embodiment, the second memory 303 included in the video processor 30 may further store the third information (or video processor specific information including the third information) described in the sixth embodiment. In this case, the third information may be directly output to an image processing circuit (including an AGC circuit) (not illustrated) in the video processor 30, or may be temporarily output to the information calculation unit 212 of the light source device 20, then returned to the video processor 30, and output to the image processing circuit (not illustrated). With this configuration, the memory in which the second information (or the light source device specific information including the second information) is stored and the memory in which the third information (or the video processor specific information including the third information) is stored can be integrated into one.

The seventh embodiment is suitable in a case where the light source device and the video processor are limited to only one combination (such as a case where the light source device and the video processor are integrally configured). Alternatively, the information calculation unit 212 may perform processing such as correcting the second information according to the combination of the light source device and the video processor.

Note that, in a case where the light source device and the video processor are limited to only one combination, in each of the second to fifth embodiments, the third information (or the video processor specific information including the third information) described in the sixth embodiment may be further stored in the second memory 208 included in the light source device 20. In this case, the video processor 30 may perform image processing based on the third information stored in the second memory 208.

### Eighth Embodiment

An eighth embodiment is a modification of each of the second to seventh embodiments, and is an aspect in which the first memory, the second memory, and the third memory are provided by a cloud or provided on a network.

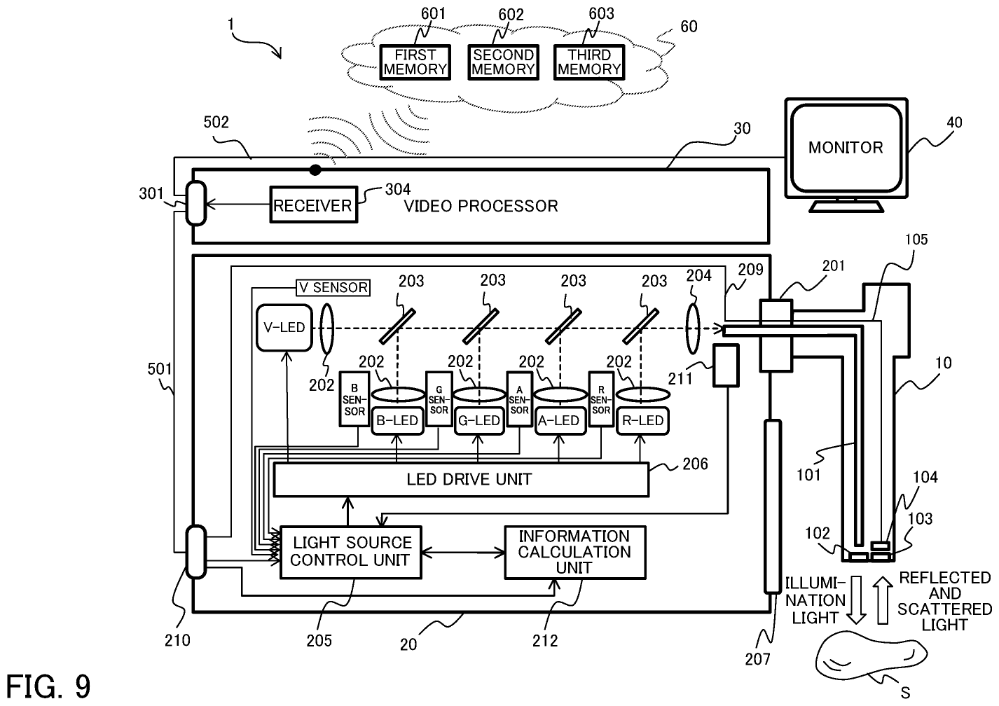

FIG. 9 is a diagram illustrating a configuration of an endoscope system according to the eighth embodiment.

In the endoscope system 1 illustrated in FIG. 9, when the scope 10, the light source device 20, and the video processor 30 are connected to each other, that is, when a combination thereof is determined, the first information and the second information, or the first information, the second information, and the third information are read from a first memory 601, a second memory 602, and a third memory 603 provided by a cloud 60. This reading may be performed by wired communication or wireless communication such as WiFi (registered trademark).

In the endoscope system 1 illustrated in FIG. 9, the video processor 30 further includes a receiver (or a transceiver) 304, and the receiver 304 reads the first information and the second information, or the first information, the second information, and the third information from the first memory 601, the second memory 602, and the third memory 603 provided by the cloud 60 by wireless communication. The read first information and second information are output to the information calculation unit 212 via the electrical connector 301, the electrical wire 501, and the electrical connector 210, and the read third information is output to an image processing circuit (not illustrated) in the video processor 30.

Note that, in the eighth embodiment, the video processor 30 includes the receiver 304, but the scope 10 or the light source device 20 may include a receiver. Alternatively, two or more of the scope 10, the light source device 20, and the video processor 30 may include a receiver. For example, in a case where each of the scope 10, the light source device 20, and the video processor 30 includes a receiver, the receiver of the scope 10 may read the first information from the first memory 601, the receiver of the light source device 20 may read the second information from the second memory 602, and the receiver of the video processor 30 may read the third information from the third memory 603. This reduces a load on each receiver. In addition, one or two of the scope 10, the light source device 20, and the video processor 30 may include a memory as in the second to seventh embodiments, and the rest may use a memory provided by the cloud 60 instead of including a memory. In this case, for example, the information calculation unit 212 may derive the combination information based on the first information read from the first memory 601 provided by the cloud 60 and the second information read from the second memory 208 of the light source device 20.

The first information, the second information, and the third information stored in the first memory 601, the second memory 602, and the third memory 603 provided by the cloud 60 may be managed by, for example, a manufacturer of the endoscope system 1, and may be appropriately updated as necessary. Accordingly, the user can operate the endoscope system 1 according to the latest first information, second information, and third information regardless of a purchase time.

The first memory 601, the second memory 602, and the third memory 603 provided by the cloud 60 may be user-dedicated memories. Accordingly, the user can operate the held endoscope system according to unified information.

In addition, the first information, the second information, and the third information may be stored in an information management system in a hospital. In this way, it is possible to centrally manage the endoscope-related information, and it is excellent in terms of information maintenance and security.

### Ninth Embodiment

A ninth embodiment is a modification of each of the second, fourth, and fifth embodiments, and the first information and the second information are different aspects.

In the ninth embodiment, the first information is information on the ratio (P.S.limit.s/P.S.limit.ss) of the upper limit value (P.S.limit.s) of the amount of light that can be emitted from the scope to the upper limit value (P.S.limit.ss) of the amount of light that can be emitted from the reference scope. The second information is information on the upper limit value (P.BS.limit.ss) of the amount of light emitted from the light source device 20 that satisfies the upper limit value (P.S.limit.ss) of the amount of light that can be emitted from the distal end of the scope of the reference scope. For example, P.S.limit.s/P.S.limit.ss is a ratio [%], and P.B.limit.ss is a light amount [W].

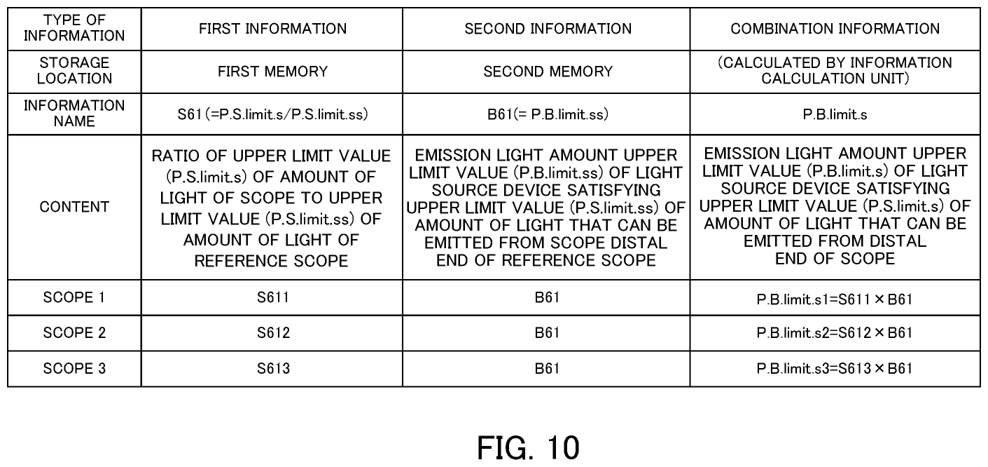

FIG. 10 is a diagram illustrating the first information, second information, and combination information according to the ninth embodiment. Here, P.S.limit.s/P.S.limit.ss, which is the first information, is indicated as S61, S61 for the “scope 1” is indicated as S611, S61 for the “scope 2” is indicated as S612, and S61 for the “scope 3” is indicated as S613. Further, P.B.limit.ss which is the second information is illustrated as B61. In this example, for example, the combination information P.B.limit.s (P.B.limit.s1) in the combination of the “scope 1” and the light source device 20 is derived by the following Expression (8) using S611 for the “scope 1” and B61 for the light source device 20.

P.B.limit.s1=S611×B61 Expression (8)

Combination information P.B.limit.s (P.B.limit.s2) in the combination of the “scope 2” and the light source device 20 and combination information P.B.limit.s (P.B.limit.s3) in the combination of the “scope 3” and the light source device 20 are similarly derived.

According to the ninth embodiment, it is possible to store the first information and the second information and derive the combination information more simply.

Although each embodiment has been described above, in the above-described embodiment, the light source of the light source device is not limited to the five color LED light sources, and for example, various light sources such as a combination of an LED light source and a laser light source, a white LED light source, a discharge lamp, a filament lamp, and the like can be applied.

Furthermore, in the above-described embodiment, the video processor 30 may include the light source control unit 205, the information calculation unit 212, and the like.

Furthermore, in the above-described embodiment, the light source device and the video processor may be configured separately or integrally. When integrated, a common memory can be used as the second memory of the light source device and the third memory of the video processor.

Furthermore, in the above-described embodiment, the illumination light is not limited to normal light, and may be special light, for example. In this case, information corresponding to the observation mode may be stored as the first information, the second information, and the third information, and the combination information may be derived based on the information.

Furthermore, in the above-described embodiment, it is also possible to apply, as the video processor, a video processor that simultaneously connects a plurality of light source devices and enables use of any one of the light source devices according to a purpose. In this case, the second information on the light source device to be used or the second information on the light source device having the lowest emission light amount upper limit value is preferably used to derive the combination information.

Furthermore, in the above-described embodiment, the amount of light emitted from the light source device has been exemplified for the second information, but the present application is not limited thereto. For example, any parameter may be used as long as the parameter can uniquely determine the amount of the light, such as an instruction value of the LED driving current for emitting the amount of light, an index value that is a numbering value corresponding to the amount of light, and an output value of a light amount sensor.

Furthermore, in the above-described embodiment, the LED drive unit 206, the connection detection unit 211, the light source control unit 205, the information calculation unit 212, and the like may be realized by a circuit. In addition, the light source control unit 205, the information calculation unit 212, and the like may be realized by an application specific integrated circuit (ASIC), a field-programmable gate array (FPGA), or the like. Furthermore, the functions of the light source control unit 205, the information calculation unit 212, and the like may be implemented by a processor such as a CPU executing a program stored in a memory.

Here, problems and effects to be solved by the above-described embodiment will be supplemented.

In an example of a conventional endoscope system having compatibility, a one-generation scope, a light source device, and a video processor are required to have compatibility of five generations in total including past two generations and future two generations. In addition, the number of types of scopes is 25 (per generation) in total including 8 types for an upper digestive organ such as the stomach and the duodenum, 9 types for a lower digestive organ such as a large intestine and the small intestine, and 8 types for other ultrasonic waves and bronchi. Furthermore, the number of types of the light source device and the video processor is two (per generation) depending on the country of sale or the like. There are seven types of observation modes: a WLI mode, an NBI mode, a red dichromatic imaging (RDI) mode, a texture and color enhancement imaging (TXI) mode, an autofluorescence imaging (AFI) mode, an enlarged WLI mode, and an enlarged NBI mode. In this example, considering all combinations, it is necessary to assume a maximum of 5×25×2×7=1,750 combinations. Of course, not all combinations are possible, but there are control parameters (control information) for about 1,000 combinations as an order.

In this example, the light source device and the video processor need to correspond to a scope of future two generations. Meanwhile, for example, even when an attempt is made to respond by a conventional method in which a control value for each scope is stored in advance in a memory included in a light source device, it is difficult to assume a specification of a scope (a scope of a future generation) to be developed in the future. In addition, there is a case where a specification change (change of an illumination optical system, correction of a heat dissipation function, or the like) is made even in a scope of a past generation, and it is also difficult to predict the change. Furthermore, there is a possibility that a new observation mode is added to the light source device, and it is also difficult to predict the new observation mode in advance and set the control parameter.

According to the above-described embodiment, for example, when the scope stores the first information regarding the scope, and the light source device stores the second information regarding the light source device, the combination information can be derived, and optimal control can be realized. In addition, even with respect to the specification change, for example, when the scope after the specification change stores the first information regarding the scope after the specification change, and the light source device after the specification change stores the second information regarding the light source device after the specification change, there is no risk of setting an erroneous control parameter (a difference in version or the like), and optimal control can be realized.

In addition, in this example, there are control parameters for about 1,000 combinations as an order, and there is a possibility that the observation mode further increases. Meanwhile, for example, when all the control parameters are held as a table, and the control is performed in association with the combination of the scope and the light source device by reading and using the combination of the scope and the light source device, an enormous time and man-hours are required for memory maintenance management and debugging. In addition, since a risk of causing a human error or a system error such as an error in association or erroneous recording increases, and there is a possibility of causing a malfunction, malfunction countermeasures, error management, and the like also increase. Furthermore, it is necessary to use a large and expensive memory, and a large-scale data management circuit is also required.

According to the above-described embodiments, for example, when the scope stores the first information regarding the scope, and the light source device stores the second information regarding the light source device, the combination information can be derived, and it is not necessary to prepare and continuously manage enormous information in advance. In addition, since the number of data to be handled is reduced by two or more digits, the risk of human error and system error is reduced, and measures, error management, and the like can be simplified. Furthermore, a small and low-cost memory can be used as the memory, and a system that handles the light source control information can also be simplified. In addition, it is possible to realize optimum control.

In addition, the scope and the light source device may have individual variations (manufacturing variations) related to specifications. Meanwhile, in the above-described embodiments, for example, it is also possible to perform more accurate and optimal light source control by measuring own variation information at the time of factory shipment inspection or the like and reflecting the variation information in the first information and the second information to store and manage the variation information. In this case, the variation information may be reflected in the first information and the second information, or may be further included in the first information and the second information as the correction value.

In addition, the value of the specification item may change depending on the use time of the scope or the light source device, the environmental temperature, and the like. The temporal change with use time tends to depend on specification items in many cases. For example, it is known that a light amount of an LED with respect to a driving current decreases at a predetermined ratio with a change with time. Therefore, by correcting the first information and the second information depending on the use time, more optimal LED control information can be obtained. In addition, since a change in the scope transmittance, yellowing of the optical system, and the like also tend to be constant, it is also possible to appropriately control the maximum light amount and the color balance by appropriately correcting the first information and the second information.

As described above, the present application is not limited to the above embodiments as they are, and in the implementation stage, the constituent elements can be modified and embodied without departing from the gist thereof. Various modifications can be made with any appropriate combinations of a plurality of constituent elements disclosed in the above embodiments. For example, some of all the components illustrated in the embodiments may be deleted. Furthermore, components in different embodiments may be combined as appropriate.

What is claimed is:

1. An endoscope device comprising a processor configured to:
calculate light source control information based on first information regarding a scope and second information regarding a light source device; and
control a light source of the light source device based on the light source control information,
wherein the second information includes a ratio of an amount of light emitted from a distal end of a reference scope to an amount of light emitted from the light source device to the reference scope.

2. The endoscope device according to claim 1, wherein
the first information includes at least one of a transmittance of a light guide optical system of the scope, an upper limit value of an amount of light that can be emitted from a distal end of the scope, and a light receiving sensitivity of an imaging element of the scope.

3. The endoscope device according to claim 1, wherein
the first information includes an upper limit value of an amount of light that can be emitted from a distal end of the scope and a ratio of transmittance of a light guide optical system of the scope to transmittance of a light guide optical system of the reference scope.

4. The endoscope device according to claim 3, wherein
the processor is configured to calculate, as the light source control information, an emission light amount upper limit value of the light source device that satisfies an upper limit value of the amount of light that can be emitted from the distal end of the scope based on the first information and the second information.

5. The endoscope device according to claim 2, wherein
the first information includes the upper limit value of the amount of light that can be emitted from the distal end of the scope, for each of a plurality of observation modes,
the processor is configured to perform the calculation until the observation mode is switched from one of the plurality of observation modes to an other of the plurality of observation modes, and
the calculation is performed using, as the upper limit value of the amount of light that can be emitted from the distal end of the scope, the upper limit value of the amount of light that can be emitted from the distal end of the scope according to the other observation mode.

6. The endoscope device according to claim 1, wherein
the second information includes the ratio of the amount of light emitted from the distal end of the reference scope to the amount of light emitted from the light source device to the reference scope, for each of a plurality of observation modes,
the processor is configured to perform the calculation until the observation mode is switched from one of the plurality of observation modes to an other of the plurality of observation modes, and
the calculation is performed using, as the ratio of the amount of light emitted from the distal end of the reference scope to the amount of light emitted from the light source device to the reference scope the ratio of the amount of light emitted from the distal end of the reference scope to the amount of light emitted from the light source device to the reference scope according to the other observation mode.

7. The endoscope device according to claim 2, wherein
the first information includes the light receiving sensitivity of the imaging element of the scope,
the second information includes a light receiving sensitivity of a standard imaging element and a predetermined color balance, and
the processor is configured to calculate, as the light source control information, a color balance of an emission light amount of the light source device based on the first information and the second information.

8. The endoscope device according to claim 7, wherein
the first information includes the light receiving sensitivity of the imaging element of the scope, for each of a plurality of observation modes,
the second information includes the light receiving sensitivity of the standard imaging element and the color balance, for each of the plurality of observation modes,
the processor is configured to perform the calculation until the observation mode is switched from one of the plurality of observation modes to an other of the plurality of observation modes, and
in the calculation, the color balance of the emission light amount of the light source device is calculated based on the light receiving sensitivity of the imaging element of the scope according to the other observation mode, and the light receiving sensitivity of the standard imaging element and the color balance according to the other observation mode.

9. The endoscope device according to claim 2, wherein
the first information includes a value obtained by dividing the upper limit value of the amount of light that can be emitted from the distal end of the scope by a ratio of the transmittance of the light guide optical system of the scope to the transmittance of the light guide optical system of the reference scope, as the information regarding the upper limit value of the amount of light that can be emitted from the distal end of the scope and a ratio of the transmittance of the light guide optical system of the scope to the transmittance of the light guide optical system of the reference scope.

10. The endoscope device according to claim 2, wherein
the first information further includes a correction factor associated with a circumstance of the scope with respect to the upper limit value of the amount of light that can be emitted from the distal end of the scope.

11. The endoscope device according to claim 1, wherein
the first information includes a ratio of an upper limit value of the amount of light that can be emitted from the scope to an upper limit value of the amount of light that can be emitted from a reference scope,
the second information includes an emission light amount upper limit value of the light source device that satisfies an upper limit value of an amount of light that can be emitted from a distal end of the reference scope, and
the processor is configured to calculate, as the light source control information, an emission light amount upper limit value of the light source device that satisfies the upper limit value of the amount of light that can be emitted from the distal end of the scope based on the first information and the second information.

12. The endoscope device according to claim 2, wherein
the first information includes information regarding a manufacturing variation of the scope, and
the second information includes information regarding a manufacturing variation of the light source device.

13. The endoscope device according to claim 2, wherein
the first information is corrected according to a use time of the scope, and
the second information is corrected according to a use time of the light source device.

14. The endoscope device according to claim 2, further comprising:
a video processor configured to perform image processing on a video signal from the scope based on third information including an upper limit value of a gain at the time of image processing for each scope.

15. The endoscope device according to claim 2, wherein
the first information is stored in a first memory included in the scope, and
the second information is stored in a second memory included in the light source device or a video processor.

16. The endoscope device according to claim 14, wherein
the first information is stored in a first memory included in the scope,
the second information is stored in a second memory included in the light source device, and
the third information is stored in a third memory included in a video processor.

17. The endoscope device according to claim 14, wherein
the first information is stored in a first memory included in the scope, and
the second information and the third information are stored in a second memory included in the light source device or a video processor.

18. The endoscope device according to claim 2, wherein
at least one of the first information and the second information is stored in a memory provided by cloud computing or a memory on a network.

19. The endoscope device according to claim 14, wherein
at least one of the first information, the second information, and the third information is stored in a memory provided by cloud computing or a memory on a network.

20. A light source device comprising a processor configured to:
calculate light source control information based on first information regarding a scope and second information regarding the light source device; and
control a light source of the light source device based on the light source control information,
wherein the second information includes a ratio of an amount of light emitted from a distal end of a reference scope to an amount of light emitted from the light source device to the reference scope.

21. An endoscope system including a scope and a light source device, the endoscope system comprising a processor configured to:
calculate light source control information based on first information regarding the scope and second information regarding the light source device; and
control a light source of the light source device based on the light source control information,
wherein the second information includes a ratio of an amount of light emitted from a distal end of a reference scope to an amount of light emitted from the light source device to the reference scope.

22. A light source control method comprising:
calculating light source control information based on first information regarding a scope and second information regarding a light source device; and
controlling a light source of the light source device based on the light source control information,
wherein the second information includes a ratio of an amount of light emitted from a distal end of a reference scope to an amount of light emitted from the light source device to the reference scope.
